(19) United States
(12) Patent Application Publication
(10) Pub. No.: US 2022/0226311 A1
(43) Pub. Date: Jul. 21, 2022

(54) PROCASPASE-3 ACTIVATION AND IMMUNOTHERAPY FOR TREATMENT OF CANCER

(71) Applicant: The Board of Trustees of the University of Illinois, Urbana, IL (US)

(72) Inventors: Paul J. HERGENROTHER, Champaign, IL (US); Timothy M. FAN, Mahomet, IL (US); Matthew BOUDREAU, Urbana, IL (US); William MONTGOMERY, Dallas, TX (US); Hyang-Yeon LEE, Savoy, IL (US); Marlies HAGER, Lincoln, NE (US); Diana RANOA, Champaign, IL (US); Myung-ryul LEE, Savoy, IL (US)

(73) Assignee: The Board of Trustees of the University of Illinois, Urbana, IL (US)

(21) Appl. No.: 17/615,402

(22) PCT Filed: Jun. 1, 2020
(86) PCT No.: PCT/US2020/035578

§ 371 Date: Nov. 30, 2021

Related U.S. Application Data
(60) Provisional application No. 62/944,404, filed on Dec. 6, 2019.
(60) Provisional application No. 62/854,823, filed on May 30, 2019.

(51) Int. Cl. A61K 31/495; A61K 39/00; A61K 39/395; A61P 35/00; A61K 9/20; A61K 9/48; A61K 9/00; A61K 47/02; A61K 9/08; A61K 47/10; A61K 47/12; A61K 9/06; A61K 47/14; A61K 47/38; A61K 47/26; A61K 47/44

(52) U.S. Cl. CPC A61K 31/495; A61K 47/44; A61K 39/3955; A61P 35/00; A61K 9/2018; A61K 9/2027; A61K 9/2054; A61K 9/2013; A61K 9/2059; A61K 9/485; A61K 9/4866; A61K 9/4858; A61K 9/0019; A61K 47/02; A61K 9/08; A61K 47/10; A61K 9/0078; A61K 47/12; A61K 9/0014; A61K 9/06; A61K 47/14; A61K 47/38; A61K 47/26; A61K 39/0011

(57) ABSTRACT

The blood-brain barrier penetrant procaspase-3-activating drug, PAC-1, has been identified as an effective approach to inducing immune stimulatory destruction of cancer cells. PAC-1 induces cleavage of MLH1 in cancer cells, and studies show that inactivation of MLH1 leads to increased mutational burden and neoantigen presentation by major histocompatibility complex (MHC) products. Herein is described a mechanistic-based strategy to bring the power of immunotherapy in an effective fashion for treatment of cancer.

### RELATED APPLICATIONS

This application claims priority under 35 U.S.C. § 119(e) to U.S. Provisional Patent Application Nos. 62/854,823 filed May 30, 2019 and 62/944,404 filed Dec. 6, 2019, which applications are incorporated herein by reference.

### GOVERNMENT SUPPORT

This invention was made with government support under Grant No. R01 CA120439 awarded by the National Institutes of Health. The government has certain rights in the invention.

### BACKGROUND OF THE INVENTION

The successful application of immunotherapeutic modalities has transformed the treatment of melanoma, lung cancer, and bladder cancer and holds considerable promise for several other tumor types. The dramatic results seen in a subset of patients—for example, durable responses even in late-stage disease—has raised hope that such treatments can be successful for other recalcitrant cancers. There exists an apparent correlation between mutational load in a tumor and success with immune checkpoint inhibitors. This observation has been successfully translated with the approval of pembrolizumab (Keytruda, an antibody to PD-1) for patients with DNA mismatch repair deficiencies or microsatellite instability (MSI). Unfortunately, for the many cancers with a low mutational load, immunotherapy trials have largely been disappointing, and the fact that less than 10% of cancers have the MSI phenotype demonstrates the challenge in broadly expanding immunotherapeutic success. The challenges of immunotherapy for glioblastoma (GBM) are even more significant, given low neoantigen expression, a lack of T-cell infiltration into the tumor, and the difficulty most drugs have in traversing the blood-brain barrier (BBB).

An important recent advance was the clinical approval of DNA microsatellite instability (MSI) as a biomarker for clinical efficacy of PD-1 inhibition (with pembrolizumab) regardless of the origin of tumorigenesis. This approval was based on considerable preclinical and clinical data showing that mismatch-repair deficiency (dMMR) predicts response of solid tumors to PD-1 blockade, as it is known that tumors with dMMR/MSI have 100s to 1000s of somatic mutations (10-fold higher than MMR-proficient cancers, FIG. 1A), presumably leading to elevated levels of neoantigens. However dMMR/MSI is present only in a low percentage of cancers, likely less than 10%, including <5% of GBM. Sporadic MSI is driven by epigenetic silencing of the MLH1 promoter, and MLH1 silencing is commonly used as a marker of MMR deficiency. The correlation between MLH1 silencing and number of somatic mutations has been demonstrated in a number of studies and is powerfully shown in FIG. 1.

Importantly, a recent report (Germano, G., et al., Nature 2017, 552, 116) has validated that inactivation of MLH1 (via CRISPR/Cas9 knockout) leads to higher mutational burden and an increased neoantigen profile. This MLH1-knockout-induced phenotype leads to cancer cells that minimally establish syngeneic tumors in mice, suggesting that dMMR is sufficient to enhance immune responses. Further, genomic MLH1-knockout leads to dramatic increases in response to immune checkpoint inhibitors (i.e. anti-PD-1+anti-CTLA-4). These results suggest that loss of MLH1 function leads to a phenotypic change driven by increased mutational load, ultimately leading to higher neoantigen expression, immune recognition, and increased sensitivity to immune checkpoint blockade in vivo.

If MLH1 loss-of-function could be induced selectively in cancer cells this could substantially elevate patient response to immunotherapies including checkpoint inhibitors and neoantigen peptide vaccines. Provocatively, multiple large proteomic studies have revealed that MLH1 is a top substrate for caspase-3, with 0% of the protein remaining after 6 hr. Further, MLH1 is only a substrate for active caspase-3 with no proteolysis observed with other active caspases (i.e. caspase-1,2,6,7,8).

The cleavage of procaspase-3 (PC-3) to caspase-3 represents a critical node in apoptosis, as this executioner caspase catalyzes the hydrolysis of hundreds of protein substrates, leading to cell death. A hallmark of cancer is the ability of tumor cells to evade apoptosis through mutation and dysregulation of apoptotic proteins, and several anticancer drug discovery strategies have focused on the inhibition of these mutated proteins. A complementary approach involves the small molecule-mediated activation of proapoptotic proteins, such as PC-3. Based on the downstream location of PC-3 in the apoptotic cascade relative to frequently mutated proteins, the low frequency of PC-3 mutations in cancer, and the robust expression of the procaspase-3 enzyme in a number of cancer types, including lymphoma, leukemia, multiple myeloma, melanoma, glioblastoma (GBM), pancreatic cancer, liver cancer, non-small cell lung cancer (NSCLC), breast cancer, ovarian cancer colon cancer, osteosarcoma, and meningioma, the small molecule-mediated activation of PC-3 is actively being explored as an anticancer strategy.

The problem is existing immunotherapy approaches to treating cancer can lack efficacy when neoantigen expression is low. Accordingly, there is a need for a drug that can selectively target cancer cells to increase their neoantigen expression, so immunotherapy treatment can help eradicate the cancer cells.

### SUMMARY

The selective activation of procaspase-3 to caspase-3 in tumors leads to quantitative cleavage of MLH1, resulting in dMMR/MSI, thereby markedly increasing the efficacy of immunotherapies. PAC-1 is used herein to selectively induce immune stimulation in cancer, including MLH1 cleavage that convert MSS tumors to dMMR/MSI tumors, thus making tumors more susceptible to treatment with immunotherapies. Results suggested that immune stimulation with PAC-1 promotes induction of a stress response, thereby altering the tumor microenvironment to increase the extent of immune inflammation. Such results bring the power of immunotherapy—dramatic and durable responses—to a greater number of cancer patients.

Accordingly, this disclosure provides a composition comprising:
(a) a procaspase-3 activator;
(b) at least one second active agent, wherein the second active agent is a check-point inhibitor, cancer vaccine, metabolic modulator, macrophage inhibitor, or immune-stimulator or modulator; and
(c) optionally a pharmaceutically acceptable diluent, excipient, or carrier.

In various embodiments, the procaspase-3 activator is PAC-1:

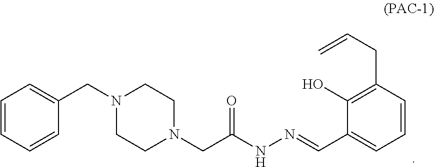 [SMILES: C=CCc1cccc(/C=N/NC(=O)CN2CCN(Cc3ccccc3)CC2)c1O]

This disclosure also provides a method of treating a cancer comprising administering to a subject in need thereof, concurrently or sequentially, a therapeutically effective amount of a procaspase-3 activator and an effective amount of a second active agent, wherein the second active agent is an immunotherapeutic; wherein the effect of the second active agent is enhanced by the administration of the procaspase-3 activator.

One certain embodiment of a method of treating cancer comprises administering to a subject PAC-1 and an anti-PD-1 antibody wherein the PAC-1 is administered daily for 21 or more consecutive days such that a total administered dose per day of the PAC-1 is about 100 mg/kg to about 125 mg/kg and the anti-PD-1 antibody is administered two times or four times over the 21 or more consecutive days, wherein a dose of the anti-PD-1 antibody is about 10 mg/kg and each of the dose of the anti-PD-1 antibody are administered on separate days.

The disclosure also provides for the use of the compositions described herein for use in medical therapy. The medical therapy can be treating cancer, for example, breast cancer, triple negative breast cancer, ovarian cancer, lung cancer, endometrial cancer, pancreatic cancer, prostate cancer, lymphoma, melanoma, leukemia, multiple myeloma, glioblastoma, liver cancer, non-small cell lung cancer, osteosarcoma, meningioma, renal cancer, metastatic renal cell carcinoma, thyroid cancer, or colon cancer. Embodiments of the disclosure also provide for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the disclosure. In some instances, embodiments of the disclosure can be best understood by referring to the accompanying drawings in combination with the detailed description presented herein. The description and accompanying drawings may highlight a certain specific example, or a certain aspect of the disclosure. However, one skilled in the art will understand that portions of the example or aspect may be used in combination with other examples or aspects of the disclosure.

FIG. 1. A) Microsatellite Instability (MSI) and B) MLH1-silencing are strongly correlated with increased numbers of somatic mutations, shown here for colon cancer in data from Vogelstein and co. (Proc Natl Acad Sci USA 2015, 112, 118). MSS, Microsatellite Stable.

FIG. 2. The synergistic effect of PAC-1 plus immunotherapy. PAC-1-induced caspase-3 cleaves certain proteins that sensitize cancer to various immunotherapy approaches.

FIG. 3. PAC-1 treatment leads to MLH1 cleavage in the absence of Apoptotic Death Markers. Cell lines were incubated with indicated concentrations of PAC-1 for 72 hours, followed by western blot analysis for MLH1 protein level, as well as PARP-1, cleaved PARP-1 (c-PARP-1 is an apoptosis maker), and beta-actin (loading control). The type of cell line is denoted with the normal cell line, HFF-1 specifically highlighted, demonstrating cancer cell specific MLH1 cleavage.

FIG. 4. PAC-1 treatment of mice with syngeneic tumors increases the numbers of tumor infiltrating lymphocytes. A) C57BL/6 mice with orthotopic transplant of GL261 neurospheres. Tumors were allowed to establish for 10 days, then mice (n=3/group) were treated with or without PAC-1 (100 mg/kg PO×10 days), then sacrificed. Tumors were stained for CD3 (brown) to identify T cell TILs. Data presented as average CD3⁺ per 4 HPF/mouse. Magnification 100×. B) C57BL/6 mice with subcutaneous transplant of B16F10 cells. Tumors were allowed to establish for 7 days, then mice (n=8/group) were treated with or without PAC-1 (100 mg/kg IP×2×14 days), then sacrificed. Tumors were stained for CD3 (brown) to identify T cell TILs. Data presented as average CD3⁺ per 10 HPF/mouse. Magnification 100×.

FIG. 5. Validation of PD-L1 and MLH1 for IHC studies. Positive PD-L1 expression in (A) human tonsil and (B) canine lymph node. Canine glioma (C) H&E and (D) PD-L1 IHC. Nuclear MLH1 IHC for (E) human U87 and (F-H) 3 canine glioma cell lines.

FIG. 6. Graph showing the efficacy of PAC-1 in combination with immunotherapy. PAC-1 dosing is at 100 mg/kg once per day. 1=Vehicle+isotope; 2=vehicle+anti-PD-1+ and-CTLA-4; 3=PAC-1+isotope; 4=PAC-1+anti-PD-1+anti-CTLA-4.

FIG. 7. Graph showing PAC-1 in combination with anti-PD-1 antibody leads to extended survival in a late-stage K7M2 metastatic model. MST=median survival time. In the figure, αPD1=anti-PD-1.

FIG. 8. Development of CT-26_WT subcutaneous model_in BALB/c mice.

FIG. 9. Growth of CT-26_WT in BALB/c mice after 2 doses (A) versus 4 doses (B). Open circle=vehicle+anti-IgG2A antibody; square=PAC-1 (100 mg/kg)+anti-IgG2a antibody; triangle=vehicle+anti-PD-1 mAb; inverted triangle=PAC-1 (100 mg/kg)+anti-PD-1 mAb.

FIG. 10. Analysis of treatment of BALB/c mice with PAC-1 and anti-PDL1 mAb. Open circle=vehicle+anti-IgG2A antibody; square=PAC-1 (100 mg/kg)+anti-IgG2a antibody; triangle=vehicle+anti-PD-1 mAb; inverted triangle=PAC-1 (100 mg/kg)+anti-PD-1 mAb.

FIG. 11. Development of CT-26_TdTomato subcutaneous tumor model in BALB/C mice.

FIG. 12. A) Example treatment protocol. B) Cytokine array of plasma from BALB/c mice treated with PAC-1.

FIG. 13. Analysis of neutrophil and macrophage populations after treatment with PAC-1 14 days post-tumor challenge in lungs, PBMC, and spleen. Open circle=vehicle+anti-IgG2A antibody; square=PAC-1+anti-IgG2a antibody; triangle=vehicle+anti-PD-1 mAb; inverted triangle=PAC-1+anti-PD-1 mAb.

FIG. 14. Analysis of T-cells, B-cells, and NK cell populations in the lungs, PBMC, and spleen of BALB/c mice 26 days post combinatorial PAC-1 and anti-PD-1 treatment. Open circle=vehicle+anti-IgG2A antibody; square=PAC-1+anti-IgG2a antibody; triangle=vehicle+anti-PD-1 mAb; inverted triangle=PAC-1+anti-PD-1 mAb.

FIG. 15. PD-L1 expression on the surface of dendritic cells and CD45⁻ tumor cells 26 days post-tumor challenge in lungs, PBMC, and spleen of BALB/c mice. Open circle=vehicle+anti-IgG2A antibody; square=PAC-1+anti-IgG2a antibody; triangle=vehicle+anti-PD-1 mAb; inverted triangle=PAC-1+anti-PD-1 mAb.

FIG. 16. Development of MC38 pulmonary metastasis model in C57BL/6 mice. 1=vehicle; 2=PAC-1; 3=anti-PD-1; 4=PAC-1+anti-PD-1. PAC-1 was delivered via intraperitoneal injection, dose of 100 mg/kg and anti-PD-1 was delivered via intraperitoneal injection, dose of 10 mg/kg.

FIG. 17. Survival curve according to the MC38 pulmonary metastasis model. 1=vehicle; 2=PAC-1; 3=anti-PD-1; 4=PAC-1+anti-PD-1. PAC-1 was delivered via intraperitoneal injection, dose of 100 mg/kg and anti-PD-1 was delivered via intraperitoneal injection, dose of 10 mg/kg.

### DETAILED DESCRIPTION

Disclosed herein is the development of a novel mechanism-based strategy to selectively convert low mutational load tumors to ones with a high mutational burden, rendering them ideal candidates for immunotherapy treatment. This strategy is premised on the targeted inactivation of the tumor suppressor MLH1. As described herein, there is a strong correlation between MLH1 silencing and response to anti-PD-1 antibodies: the link between genetic silencing of MLH1 and the number of somatic mutations in a tumor has been convincingly demonstrated, and the DNA damage resulting from MLH1 loss of function elicits a highly immunogenic stress response. The goal was to bring the power and potential of immunotherapy to GBM through drug-mediated, tumor-selective inactivation of MLH1. MLH1 is a major cellular substrate for caspase-3, and the disclosed method can induce selective MLH1 cleavage in cancer cells with a small molecule called PAC-1 that selectively activates procaspase-3 to caspase-3 in tumor cells.

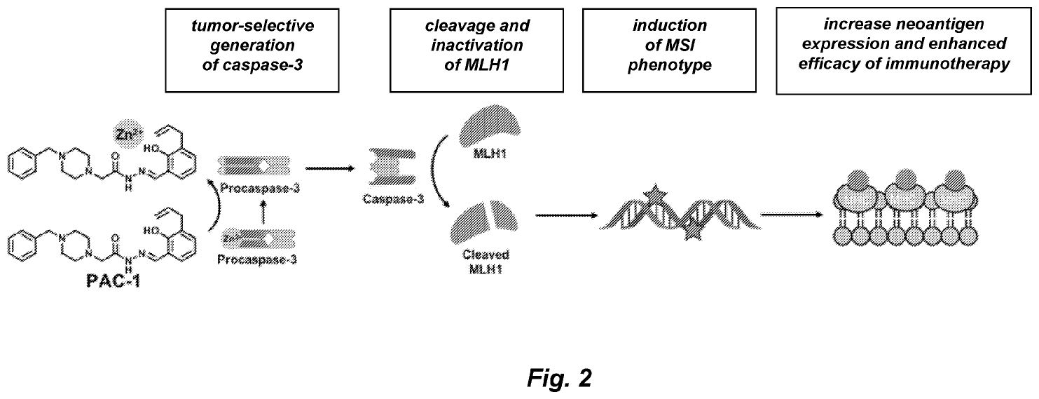

PAC-1 is an orally available, BBB penetrant experimental therapeutic that has proven safe in human cancer patients and is currently being evaluated clinically (in combination with radiation and temozolomide) for GBM. The overall objective of this application is to achieve mechanism-based synergies of drug-induced MLH1 cleavage with immunotherapies in sophisticated models of GBM. The central hypothesis was that drug mediated MLH1 cleavage will induce tumor selective DNA damage and MSI, thus increasing the quantity (and immunogenicity) of potential neoantigens. Furthermore, the caspase-3 inducing activity of PAC-1 promotes an inflammatory intratumoral environment, thus turning ‘cold’ GBM tumors to ‘hot’ tumors that are vulnerable to various immunotherapy modalities (FIG. 2).

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the disclosure encompasses not only the entire group listed as a whole, but each member of the group individually and all possible subgroups of the main group. Additionally, for all purposes, the disclosure encompasses not only the main group, but also the main group absent one or more of the group members. The disclosure therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping, or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

The compositions described herein may be administered with additional compositions to prolong stability and activity of the compositions, or in combination with other therapeutic drugs. The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

The term “immunotherapy” refers to the treatment of disease by activating or suppressing the immune system with, for example, an “immunotherapeutic”. Immunotherapies designed to elicit or amplify an immune response are classified as activation immunotherapies, while immunotherapies that reduce or suppress are classified as suppression immunotherapies. Immunotherapy is the treatment of disease by activating or suppressing the immune system. These immunotherapies are designed to elicit or amplify an immune response are classified as activation immunotherapies, while immunotherapies that reduce or suppress are classified as suppression immunotherapies. Cancer immunotherapy attempts to stimulate the immune system to destroy tumors.

The term “isotype” refers to controls that are primary antibodies that lack specificity to the target but match the class and type of the primary antibody used in the application. Isotype controls are used as negative controls to help differentiate non-specific background signal from specific antibody signal.

### Embodiments of the Disclosure

This disclosure provides a composition comprising:
(a) a procaspase-3 activator;
(b) at least one second active agent, wherein the second active agent is a check-point inhibitor, cancer vaccine, metabolic modulator, macrophage inhibitor, or immune-stimulator or modulator; and
(c) optionally a pharmaceutically acceptable diluent, excipient, or carrier.

In various embodiments, the procaspase-3 activator is PAC-1:

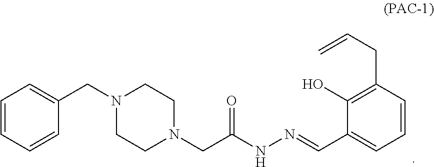 [SMILES: C=CCc1cccc(/C=N/NC(=O)CN2CCN(Cc3ccccc3)CC2)c1O]

In various additional embodiments, the procaspase-3 activator is a compound disclosed in U.S. Pat. Nos. 8,592,584; 8,778,945; 8,916,705; or 9,249,116; the formulas and compounds of which are incorporated herein by reference.

In additional embodiments, the second active agent has an effect in a cancer cell that induces apoptosis and PAC-1 enhances the effect of the second active agent by an amount greater than an additive effect, wherein PAC-1 primes the vulnerability of the cancer cell to the second active agent.

In various other embodiments, the composition (e.g., the procaspase-3 activator) suppresses mismatch-repair (MMR) proteins. In additional embodiments, the composition is a mediator of caspase-3 degradation of MutL homolog 1 (MLH1) proteins. In further embodiments, the composition induces DNA microsatellite instability (MSI). In yet other embodiments, the composition selectively targets cancer cells.

In some further embodiments, MMR proteins comprise MutL homolog 1 (MLH1) proteins, and wherein degradation of MMR proteins (e.g., MLH1 proteins), mediated by caspase-3 activation via a procaspase-3 activator, leads to a deficiency of MMR proteins (i.e., dMMR) and can further induce DNA microsatellite instability (MSI) and neoantigen expression, thereby enhancing the effect of the immunotherapeutic, wherein the procaspase-3 activator increases tumor-infiltrating lymphocytes in the cancer.

In other additional embodiments, the at least one second active agent is at least one check-point inhibitor that regulates an immune response via programmed cell death protein 1 (PD-1), programmed death-ligand 1 (PD-L1), cytotoxic T-lymphocyte-associated protein 4 (CTLA-4), T-cell immunoglobulin and mucin-domain containing-3 (TIM-3), lymphocyte-activation gene 3 (LAG-3), tumor necrosis factor receptor superfamily-member 4 (TNFRSF4 or OX40), tumor necrosis factor receptor superfamily-member 9 (TNFRSF9 or 4-1BB), glucocorticoid-induced TNFR-related protein (GITR), inducible T-cell costimulator (ICOS), or a combination thereof.

In various additional embodiments, the second active agent modulates indoleamine-pyrrole 2,3-dioxygenase (IDO), adenosine A_{2A} receptor (A2AR), transforming growth factor beta (TGF-β), C—X—C chemokine receptor type 4 (CXCR-4), C—C chemokine receptor type 4 (CCR4), tumor necrosis factor receptor (CD27), interleukin-2 receptor subunit beta (CD122), death receptor 5 (DR5), inhibitors of apoptosis proteins (IAP), glutaminase, colony stimulating factor 1 receptor (CSF1R), toll-like receptors (TLRs), dendritic cells (DC), or a combination thereof.

In yet further embodiments, the second active agent is ADXS11-001, ADXS31-142, AMP-224, AMP-514, atezolimumab, atezolizumab, avelumab, bevacizumab, cemiplimab, BLZ945, BMS-936559, BMS986016, BMS986156, BMS986205, CB839, CIMAvax, CMP001, CP870893, CPI-444, CRS207, CV301, DC vaccine, DNX2401, DS-8273a, durvalumab, epacadostat, FAZ053, FPA008, GDC0919, GSK3174998, GVAX, GWN323, IMCgp100, IMP321, imprime PGG, indoximid, ipilimumab, JTX-2011, LAG525, LCL161, LK-301, LY2157299, LY2510924, LY3022855, MBG453, MEDI0562, MEDI0680, MEDI6469, MEDI9447, MGN1703, mogamulizumab, MOXR0916, neoantigen vaccine, NEO-PV-01, NIS793, nivolumab, NKTR-214, PBF509, PDR001, pembrolizumab, peptide vaccine, pexidartinib (PLX3397), PF-04518600, PF-3512676, REGN2810, REGN3767, R07009789, SD101, talimogene laherparepvec, TPIV200/huFR-1, tremelimumab, TroVax, TSR022, ulocuplumab, urelumab, utomilumab, varlilumab, viagenpumatucel-L (HS-110), or a combination thereof.

In other embodiments, the checkpoint inhibitor is anti-PD-1, anti-CTLA-4, or a combination thereof; wherein the anti-PD-1 is nivolumab or pembrolizumab, the anti-CTLA-4 is ipilimumab or tremelimumab, or a combination thereof.

In some embodiments, the disclosed composition comprises a pharmaceutically acceptable diluent, excipient, carrier, or a combination thereof. In other embodiments of the disclosed composition, a) the carrier comprises water, a buffer, a sugar, a cellulose, a cyclodextrin, dimethyl sulfoxide, polyethylene glycol, tocopherol, a liposome, a micelle, or a combination thereof, or b) the excipient comprises, a binder, a lubricant, a sorbent, a vehicle, a disintegrant, a preservative, or a combination thereof.

In various other embodiments, the concentration of PAC-1 is about 0.1 μM to about 50 μM. In other embodiments, the concentration of PAC-1 is about 0.1 μM to about 1 μM, about 1 μM to about 10 μM, about 2 μM to about 15 μM, about 3 μM to about 20 μM, about 4 μM to about 25 μM, about 5 μM to about 30 μM, about 10 μM to about 40 μM, about 15 μM to about 50 μM, or about 0.01 μM to about 100 μM.

In additional embodiments, the concentration of the second active agent is about 1 nM to about 100 μM. In other embodiments, concentration of the second active agent is about 1 nM to about 100 nM, about 10 nM to about 1 μM, about 100 nM to about 1 μM, about 1 μM to about 5 μM, about 1 μM to about 10 μM, about 5 μM to about 15 μM, about 10 μM to about 20 μM, about 10 μM to about 30 μM, about 15 μM to about 40 μM, about 20 μM to about 50 μM, or about 50 μM to about 100 μM.

In further embodiments, the composition disclosed herein selectively targets cancer cells, wherein the cancer cells are cells of bladder cancer, breast cancer, colon cancer, endometrial cancer, glioblastoma, leukemia, liver cancer, lung cancer, lymphoma, melanoma, meningioma, multiple myeloma, ovarian cancer, osteosarcoma, pancreatic cancer, prostate cancer, renal cancer, or thyroid cancer; wherein the breast cancer is optionally triple negative breast cancer, lung cancer is optionally non-small cell lung cancer, and renal cancer is optionally metastatic renal cell carcinoma.

This disclosure also provides a method of inhibiting the growth or proliferation of cancer cells comprising contacting cancer cells with an effective amount of the disclosed composition, thereby inhibiting the growth or proliferation of the cancer cells. In other embodiments, the growth or proliferation of the cancer cells is inhibited by suppressing mismatch-repair (MMR) proteins. In further embodiments, the growth or proliferation of the cancer cells is inhibited by caspase-3 activation mediated degradation of MutL homolog 1 (MLH1) proteins. In yet other embodiments, DNA microsatellite instability (MSI) is induced.

This disclosure further provides a method of inducing apoptosis in a cancer cell comprising contacting the cancer cell with an effective amount of a composition disclosed herein, wherein apoptosis is thereby induced by suppressing mismatch-repair (MMR) proteins in the cancer cell. In other embodiments, degradation of MutL homolog 1 (MLH1) proteins is a mediated by caspase-3 activation via the procaspase-3 activator, thereby inducing apoptosis in the cancer cell.

Additionally, this disclosure provides a method of treating a cancer comprising administering to a subject in need thereof, concurrently or sequentially, a therapeutically effective amount of a procaspase-3 activator and an effective amount of a second active agent, wherein the second active agent is an immunotherapeutic; wherein the effect of the second active agent is enhanced by the administration of the procaspase-3 activator.

In yet other additional embodiments, the procaspase-3 activator is PAC-1, or wherein the procaspase-3 activator has a molecular weight of about 200 to about 800, about 250 to about 550, about 300 to about 600, about 350 to about 550, or about 350 to about 450, wherein the procaspase-3 activator directly activates procaspase-3 to caspase-3.

In further embodiments, the second active agent comprises a check-point inhibitor, cancer vaccine, metabolic modulator, macrophage inhibitor, immune-stimulator, or modulator; or a combination thereof.

In various embodiments, caspase-3 degradation of MutL homolog 1 (MLH1) proteins induces DNA microsatellite instability (MSI) and neoantigen expression, thereby increasing the effectiveness of cancer treatment. In other embodiments, mismatch-repair (MMR) proteins are suppressed by the procaspase-3 activator. In further embodiments, the procasepase-3 activator, for example, PAC-1, increases tumor-infiltrating lymphocytes (TILs) in the cancer (or cancer cells).

In various other embodiments, the immunotherapeutic is a check-point inhibitor, and the check-point inhibitor regulates an immune response via programmed cell death protein 1 (PD-1), programmed death-ligand 1 (PD-L1), cytotoxic T-lymphocyte-associated protein 4 (CTLA-4), T-cell immunoglobulin and mucin-domain containing-3 (TIM-3), lymphocyte-activation gene 3 (LAG-3), tumor necrosis factor receptor superfamily-member 4 (TNFRSF4 or OX40), tumor necrosis factor receptor superfamily-member 9 (TNFRSF9 or 4-1BB), glucocorticoid-induced TNFR-related protein (GITR), inducible T-cell costimulator (ICOS), or a combination thereof.

In yet other various embodiments, the second active agent is atezolimumab, avelumab, bevacizumab, BMS986016, BMS986156, CP870893, durvalumab, FAZ053, GSK3174998, GWN323, IMP321, ipilimumab, JTX-2011, LAG525, MBG453, MEDI0562, MEDI0680, MEDI6469, MOXR0916, nivolumab, PDR001, pembrolizumab, PF-04518600, REGN2810, REGN3767, R07009789, tremelimumab, TSR022, urelumab, utomilumab, or a combination thereof.

In various additional embodiments, the concentration of PAC-1 is about 0.1 μM to about 50 μM and the concentration of the second active agent is about 1 nM to about 100 μM. In further embodiments, the concentration of PAC-1 is about 1 μM to about 10 μM. In other embodiments, the concentration of the second active agent is about 1 nM to about 1 μM.

In various embodiments, as would be readily recognized by one of skill in the art, the concentrations of PAC-1 and the second active agent(s) recited throughout this disclosure can also be recited and interpreted as ratios of PAC-1 to the second active agent, for example, by converting the concentrations recited herein to their corresponding molar ratios of PAC-1 to the second active agent(s).

In various other embodiments, the cancer is bladder cancer, breast cancer, colon cancer, endometrial cancer, glioblastoma, leukemia, liver cancer, lung cancer, lymphoma, melanoma, meningioma, multiple myeloma, ovarian cancer, osteosarcoma, pancreatic cancer, prostate cancer, renal cancer, or thyroid cancer; wherein the breast cancer is optionally triple negative breast cancer, lung cancer is optionally non-small cell lung cancer, and renal cancer is optionally metastatic renal cell carcinoma.

In some various embodiments, the compound PAC-1 and the second active agent are concurrently administered to the subject. In yet other embodiments, the compound PAC-1 and the second active agent are sequentially administered to the subject. In additional embodiments, the compound PAC-1 is administered to the subject before the second active agent. In further embodiments, the compound PAC-1 is administered to the subject after the second active agent.

Furthermore, this disclosure provides a composition to prepare a medicament for use in the treatment of cancer, the composition comprising:

(a) the compound PAC-1;

(b) at least one second active agent, wherein the second active agent is a check-point inhibitor, cancer vaccine, metabolic modulator, macrophage inhibitor, or immune-stimulator or modulator; and

(c) optionally a pharmaceutically acceptable diluent, excipient, carrier, or combination thereof; wherein the cancer is thereby treated.

In additional embodiments, the concentration of PAC-1 is about 0.1 μM to about 500 μM and the concentration of the second active agent is about 1 nM to about 1000 μM. In yet other additional embodiments, the second active agent is atezolimumab, avelumab, bevacizumab, durvalumab, ipilimumab, nivolumab, pembrolizumab, tremelimumab, urelumab, utomilumab, or a combination thereof. In yet further embodiments, the cancer is lymphoma, melanoma, leukemia, multiple myeloma, glioblastoma, pancreatic cancer, liver cancer, non-small cell lung cancer, breast cancer, ovarian cancer, colon cancer, osteosarcoma, or meningioma.

In various embodiments, the compound PAC-1 and the second active agent are administered to the subject once daily (q.d.), twice a day (b.i.d.), three times a day (t.i.d.), or four times a day (q.i.d.), wherein the total administered dose per day of PAC-1 is about 1 mg/kg to about 150 mg/kg, about 10 mg/kg to about 125 mg/kg, or about 5 mg/kg to about 250 mg/kg. In other embodiments, each administered dose of PAC-1 (or second active agent) is about 10 mg, about 25 mg, about 50 mg, about 60 mg, about 70 mg, about 75 mg, about 175 mg, about 250 mg, about 375 mg, about 450 mg, about 500 mg, about 625 mg, about 750 mg, about 1000 mg, or about 10 mg to about 2000 mg. In further embodiments, each administered dose of PAC-1 (or second active agent) is about 50 mg/m² to about 250 mg/m², or about 10 mg/m² to about 500 mg/m². In some other embodiments, the daily total administered dose per day of the second active agent is about 1 mg/kg to about 100 mg/kg, or about 5 mg/kg to about 150 mg/kg.

In some embodiments, the composition administered to a patient in need of treatment for cancer comprises PAC-1 and alpha-PD-1 wherein the amount of PAC-1 administered is about 100 mg/kg to about 150 mg/kg (or about 125 mg/kg) and the amount of alpha-PD-1 administered is about 150 micrograms to about 250 micrograms (or about 200 micrograms); in various embodiments, the survival of the patient is prolonged in comparison to a control.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

### Results and Discussion

Immunotherapy involving checkpoint inhibitors has become an effective treatment for certain cancers (e,g, melanoma, NSCLC, urothelial), with the ability to induce durable responses in subsets of cancer patients. There are now dozens of on-going combination trials involving immune checkpoint inhibitors and small molecule drugs. The mechanistic hypothesis that direct procaspase-3 activation dramatically enhances the efficacy of immune checkpoint inhibitors by enhancing cleavage of the key DNA mismatch repair protein MLH1, resulting in an increase in potential neoantigens targeted by T cells has been explored, as described herein.

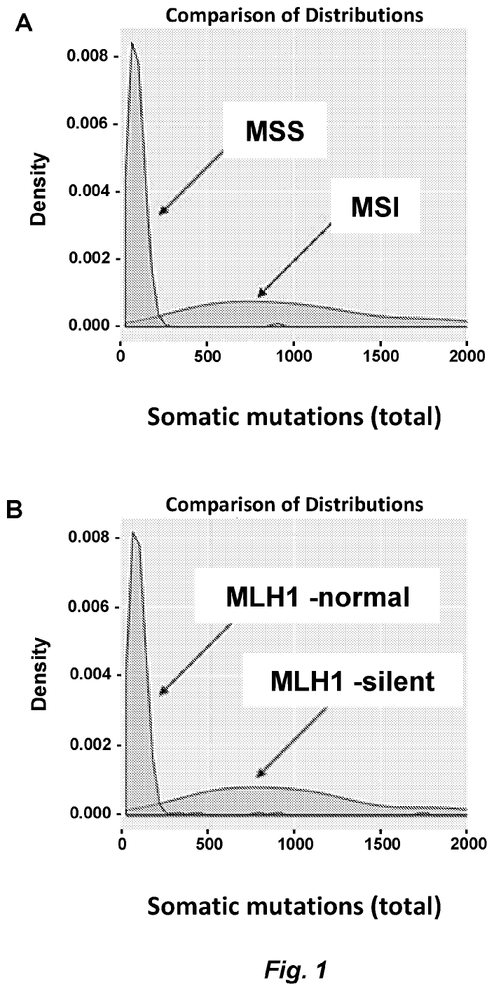

Background of approach. The considerable promise of immunotherapy involving immune checkpoint inhibitors has been tempered by low percentage of responders and failures in many clinical trials. An important recent advance was the clinical approval of DNA microsatellite instability (MSI) as a biomarker for clinical efficacy of PD-1 inhibition (with pembrolizumab) regardless of the origin of tumorigenesis. This approval was based on considerable preclinical and clinical data showing that mismatch-repair deficiency (dMMR) predicts response of solid tumors to PD-1 blockade, as it is known that tumors with dMMR/MSI have 100s-1000s somatic mutations (10-fold higher than MMR-proficient cancers, FIG. 1A), presumably leading to elevated levels of neoantigens and enhanced T cell infiltration. However, dMMR/MSI is present only in a low percentage of all cancers, likely less than 10%. Sporadic MSI is driven by epigenetic silencing of the MLH1 promoter, and MLH1 silencing is commonly used as a marker of MMR deficiency. The correlation between MLH1 silencing and number of somatic mutations has been demonstrated in a number of studies and is powerfully shown in FIG. 1.

MSI induced selectively in cancer cells substantially elevates patient response to immune checkpoint inhibitors (e.g., targeted to PD-1 and CTLA-4). Provocatively, multiple large proteomic studies have revealed that MLH1 is a top substrate for caspase-3, with 0% of the protein remaining after 6 hr (compared to MEK1/2, which have 70% remaining at the same time point). Further, MLH1 is only a substrate for active caspase-3 with no proteolysis observed with other active caspases (i.e. caspase-1,2,6,7,8). This data suggests that the selective activation of PC-3 in tumors could lead to quantitative cleavage of MLH1, resulting in dMMR/MSI, thereby markedly increasing the efficacy of immune checkpoint inhibitors; as outlined schematically in FIG. 2.

Mechanism of action. This disclosure shows that PAC-1 can be used to selectively induce MLH1 cleavage in cancers, thus making them more susceptible to treatment with immune checkpoint inhibitors (FIG. 2). Furthermore, treatment with PAC-1 induces a stress response and thereby alter the tumor microenvironment to increase the extent of immune inflammation. Such results bring the power of immunotherapy—dramatic and durable responses—to a much larger swath of cancer patients. In summary, MLH1 cleavage and inactivation by caspase-3 agonizes the innate immune system and leads to both point mutations and indels (with neoantigens derived from novel open reading frames) that will be immunogenic. Thus, this chemically induced MLH1 degradation enhances the anticancer immune response.

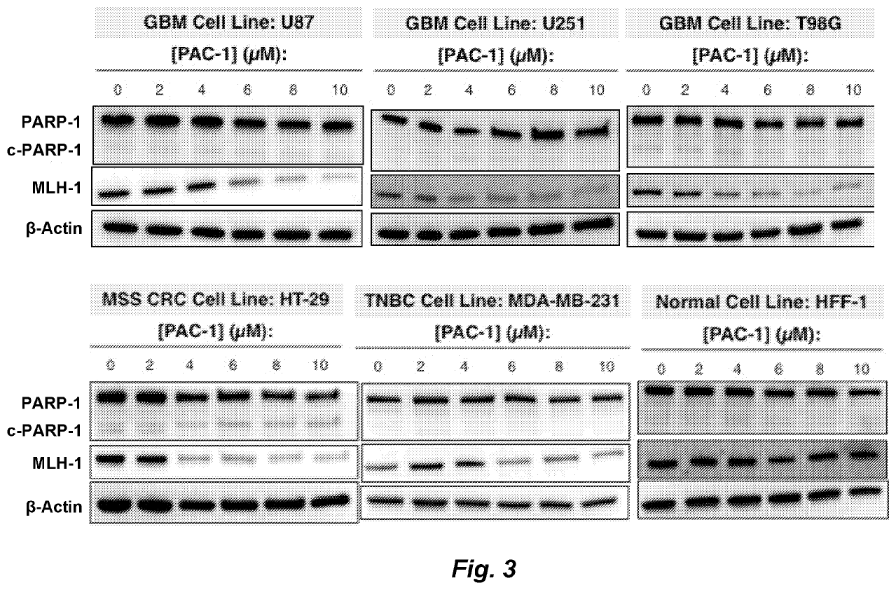

Results. MSS/MSI status of colon cancer cell lines have been reported (Ahmed, D., et al., Oncogenesis 2013, 2, e71), allowing for selection of HT-29, an MSS colon cancer cell line. To date, studies that have focused on the cleavage of MLH1 have utilized strategies that broadly induce high levels of apoptotic cell death (i.e., with staurosporine). HT-29 cells were treated with sub-lethal PAC-1. As shown in FIG. 3, PAC-1 treatment of HT-29 cells induced PC-3 activation and MLH1 cleavage, but little to no PARP-1 cleavage at these times and concentrations. This result further validates MLH1 as an outstanding substrate of caspase-3; importantly the concentrations of PAC-1 used in these experiments are sustainable in human cancer patients over a period of weeks (at 450 mg, Cₘᵢₙ=3.2 μM, Cₘₐₓ=7.8 μM).

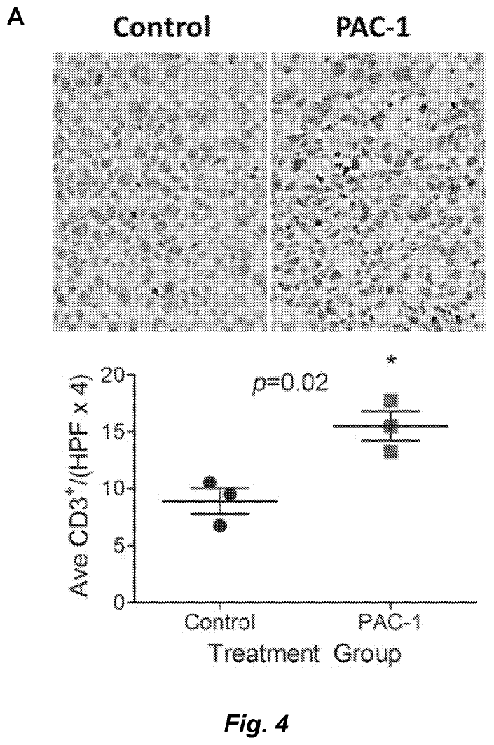

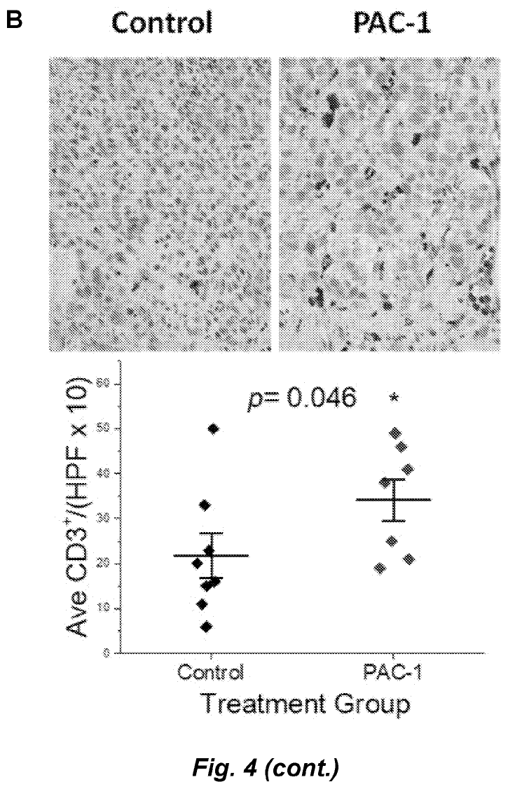

Also, an experiment in syngeneic GL261 and B16F10 mouse models that showed that single-agent PAC-1 increased the number of TILs (CD3⁺ cells) (FIG. 4) was conducted.

A large body of data on PAC-1 suggests that it does not induce cancer in vivo. This has been seen through a) treatment of pet dogs with cancer, some of whom have been treated for >6 months with PAC-1 and remain free of secondary malignancies >12 months upon completion of therapy, b) detailed IND-enabling toxicology studies in rats and research dogs, including an 84-day continuous treatment dog study, c) the data from the human clinical trial. Multiple patients have taken PAC-1 beyond the 2-month window of the trial, including 2 that have taken it for over 10 months (one at 450 mg daily), with no ill effects. It should be noted that cancer drugs can induce secondary cancers, for example, almost ⅓ of patients treated with single-agent vemurafenib develop secondary malignancies; but to date this has not been observed for PAC-1. Just as PAC-1 induces PC-3 cleavage selectively in cancer cells, the resulting caspase-3 activity should lead to selective MLH1 cleavage in cancer cells. It is worth noting that Turcot syndrome, a constitutional mismatch repair deficiency (CMMRD) cancer prone syndrome, is correlated with biallelic germline mutations in MMR genes, resulting in the development of GBM at a young age. Turcot syndrome and other CMMRD syndromes (i.e. Lynch Syndrome) point to the importance of maintaining MMR protein function, implying that inducing MLH1 cleavage/loss in a non-targeted, pan-organism fashion is not a viable therapeutic strategy. However, the strategy with PAC-1 leverages the well-known overexpression of PC-3 in cancer cells (including GBM) resulting in targeted MLH1 cleavage in tumors, leaving MMR proteins in normal cells unperturbed and operating.

### Pharmaceutical Formulations

The compounds and compositions described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium, or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical, or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose, or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Gena), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the disclosure provides a composition comprising a compound of the disclosure formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-tumor agents and have higher potency and/or reduced toxicity as compared to immunotherapies alone or other cancer treatments.

The disclosure provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine, vertebrates, and the like. Cancer refers to any various type of malignant neoplasm, for example, colon cancer, breast cancer, ovarian cancer, osteosarcoma, melanoma and leukemia, and in general is characterized by an undesirable cellular proliferation, e.g., unregulated growth, lack of differentiation, local tissue invasion, and metastasis.

The ability of a compound of the disclosure to treat cancer may be determined by using assays well known to the art. For example, the design of treatment protocols, toxicity evaluation, data analysis, quantification of tumor cell kills, and the biological significance of the use of transplantable tumor screens are known. In addition, ability of a compound to treat cancer may be determined using the Tests as described below.

The following Examples are intended to illustrate the above disclosure and should not be construed as to narrow its scope. One skilled in the art will readily recognize that the Examples suggest many other ways in which the disclosure could be practiced. It should be understood that numerous variations and modifications may be made while remaining within the scope of the disclosure.

### EXAMPLES

### Example 1. Experimental Procedure for the 4T1 Efficacy Model

Reagents. The following antibodies were purchased from Bio X Cell: anti-mouse CTLA-4 monoclonal antibody (9H10), anti-mouse PD-1 monoclonal antibody (RMP1-14), rat IgG2a isotype control (2A3), and polyclonal Syrian hamster IgG.

Cell lines. 4T1 murine breast cancer cell line was obtained from ATCC and was cultured in complete RPMI1640, containing 10% FBS, 100 U/mL penicillin, 100 μg/mL streptomycin at 37° C. in a CO₂ incubator.

4T1 orthotopic tumor model. All experimental procedures were approved by the Institutional Animal Care and Use Committee at the University of Illinois at Urbana-Champaign. 6-8 weeks old female BALB/c mice were purchased from Charles River and allowed to acclimate for 7 days. Mice were lightly sedated with i.p. xylazine (16 mg/kg) and ketamine (100 mg/kg). Following sedation, 100 μL 4T1 cells in chilled HBSS (10 million cells/mL) were injected into the right second mammary gland of the mice. The orthotopic growing tumor was established after a week. 12 Days after inoculation of 4T1 cells, tumor bearing mice were randomized into 4 treatment groups: vehicle+isotypes, vehicle+anti-PD-1/anti-CTLA-4, PAC-1+isotypes, PAC-1+anti-PD-1/anti-CTLA-4 (n=6). PAC-1 was formulated in HPβCD (10 mg/mL in 200 mg/mL HPβCD at pH 5.5).

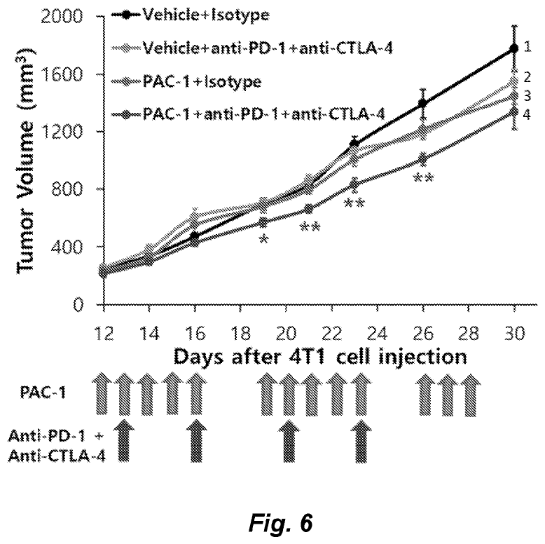

All antibodies were diluted to appropriate concentrations in sterile PBS (pH 7.0). Vehicle or 100 mg/kg PAC-1 was administered intraperitoneally for 5 consecutive days for 3 weeks. Isotypes or 10 mg/kg anti-PD-1+10 mg/kg anti-CTLA-4 antibodies were administered intraperitoneally 4 h after PAC-1 on day 13, 16, 20, and 23 post tumor implantations. Tumor measurements were performed every 2 or 3 days using a caliper and tumor volume was calculated using the equation (0.5×l×w²). On day 30 after the 4T1 cells inoculation, the mice were sacrificed. Tumors were then excised, and their mass was measured. All statistical analysis was performed using an unpaired, two-tailed student's t test with p values <0.05 were considered statistically significant (see FIG. 6).

### Example 2. Tumor Studies

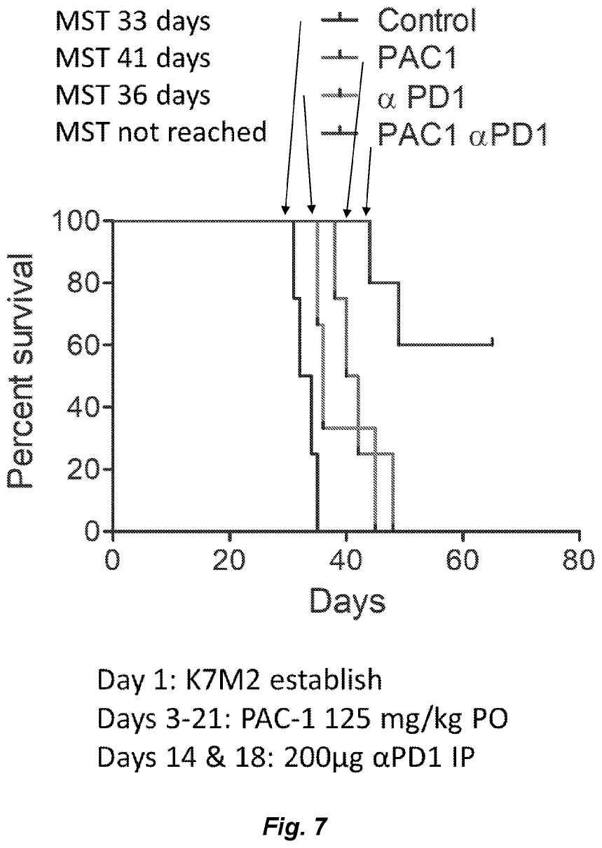

Increased immune cell infiltration in PAC-1 treated GL261 tumors. In addition to data showing that PAC-1 induces caspase-3 mediated cleavage of MLH1 in cancer cells, there are a number of other pieces of evidence that support the synergistic combination of PAC-1 with immunotherapeutic strategies (including checkpoint inhibitors and neoantigen vaccines): 1) The transcript profile of cancer cells treated with PAC-1 shows upregulation of key genes including TNFα, innate immune system agonists IL-1β and IL-8, and no upregulation of markers associated with resistance to anti-PD-1 therapy (IPRES: e.g. CCL2, CCL7, CCL8, CCL13, and others). 2) Work from another group shows PAC-1 can enhance extrinsic cell death in culture via combination studies with the immune cytokine TRAIL. 3) PAC-1 is efficacious in in vivo settings with intact immune systems, including syngeneic mouse (EL4, K7M2, GL261) (FIG. 7) and rat (9L) models, and canine cancer patients. 4) The Gandhi group at MD Anderson (Blood 2015, 125, 1126) has shown that PAC-1 and a derivative have minimal toxicity to PMBCs. 5) PAC-1 has not been observed to induce myelosuppression (in mice, rats, dogs, or humans), even when used at very high doses in the IND-enabling rat and dog studies. 6) PAC-1 causes DNA damage selectively in cancer cells, further validating studies showing caspase-3 activation can lead to significant genomic instability. As a start toward exploring the potential of PAC-1 for stimulating an immune response, an experiment in the syngeneic GL261 mouse model which showed that single-agent PAC-1 increased the number of TILs (CD3⁺ cells) (FIG. 4) was conducted.

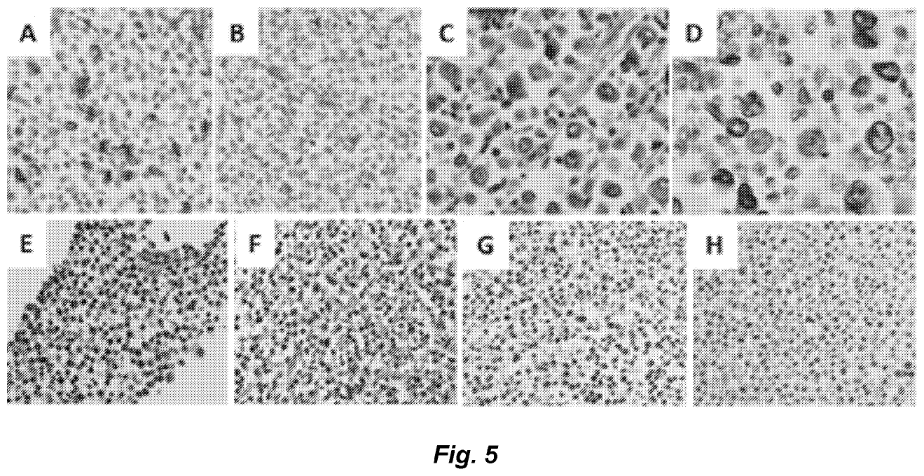

Relevance of immune checkpoints in canine glioma. Recent investigations have identified the expression of PD-L1 in various canine tumor types. However, no published studies have described PD-L1 in canine glial tumors. Using archived canine glioma tumors, the cross-reactivity of a commercial mouse monoclonal anti-human PD-L1 antibody (Abcam, clone ABM4E54) (FIG. 5) was validated, and it was demonstrated that PD-L1 was expressed in 75% of tumors; this frequency is comparable to human GBM in which PD-L1 has been identified in 88% and 72% of primary and recurrent samples, respectively. In addition to PD-L1, antibodies have been validated to be cross-reactive with the nuclear target, MLH1, in human and canine glioma cell lines, and allows one to quantitatively assess MLH1 cleavage following PAC-1 therapy.

### Example 3. PAC-1 and Immunotherapy in a Syngeneic Colon Cancer Model (CT-26 Cells)

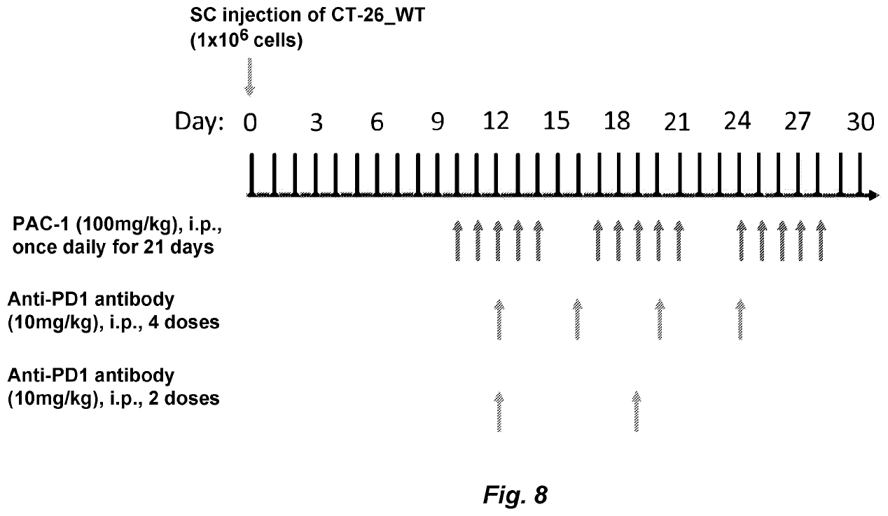

FIG. 8 illustrates the development of a CT-26_WT subcutaneous disease model in BALB/c mice. At day 0, BALB/c mice were injected subcutaneously with 1×10⁶ CT-26_WT cells. At various day intervals, selected mice were injected (i.p.) with an empty vehicle, PAC-1 (100 mg/kg), anti-PD-1 antibody (10 mg/kg; 2 doses), anti-PD-1 antibody (10 mg/kg; 4 doses), or a combination of PAC-1 (100 mg/kg) and anti-PD-1 antibody (10 mg/kg;) as shown in Table 1.

TABLE 1
Treatment protocol of BALB/c mice.

| | Ear tag # | Treatment comb'n | | # of mice |
|---|---|---|---|---|
| Group A (2x) | 6161 | vehicle | anti-IgG | n = 3 |
| | 6162 | vehicle | anti-IgG | |
| | 6163 | vehicle | anti-IgG | |
| | 6173 | PAC-1 | anti-IgG | n = 3 |
| | 6174 | PAC-1 | anti-IgG | |
| | 6175 | PAC-1 | anti-IgG | |
| | 6179 | vehicle | anti-PD1 | n = 3 |
| | 6180 | vehicle | anti-PD1 | |
| | 6181 | vehicle | anti-PD1 | |
| | 6185 | PAC-1 | anti-PD1 | n = 4 |
| | 6186 | PAC-1 | anti-PD1 | |
| | 6187 | PAC-1 | anti-PD1 | |
| | 6171 | PAC-1 | anti-PD1 | |
| Group B (4x) | 6164 | vehicle | anti-IgG | n = 3 |
| | 6155 | vehicle | anti-IgG | |
| | 6172 | vehicle | anti-IgG | |
| | 6176 | PAC-1 | anti-IgG | n = 3 |
| | 6177 | PAC-1 | anti-IgG | |
| | 6178 | PAC-1 | anti-IgG | |
| | 6182 | vehicle | anti-PD1 | n = 3 |
| | 6183 | vehicle | anti-PD1 | |
| | 6184 | vehicle | anti-PD1 | |
| | 6188 | PAC-1 | anti-PD1 | n = 3 |
| | 6189 | PAC-1 | anti-PD1 | |
| | 6190 | PAC-1 | anti-PD1 | |

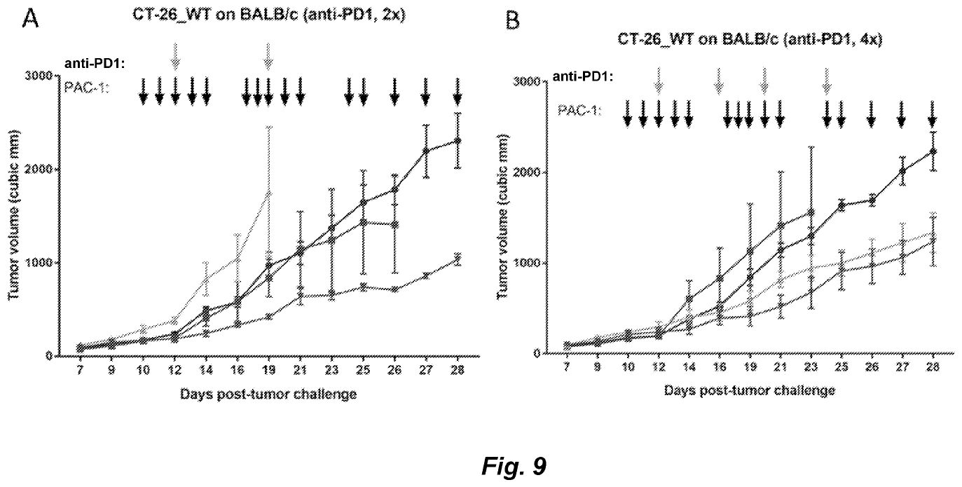

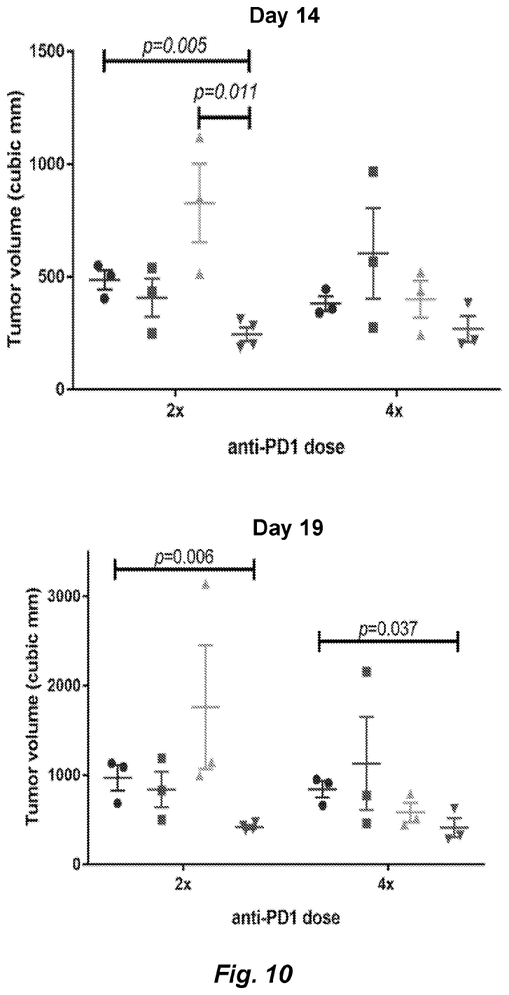

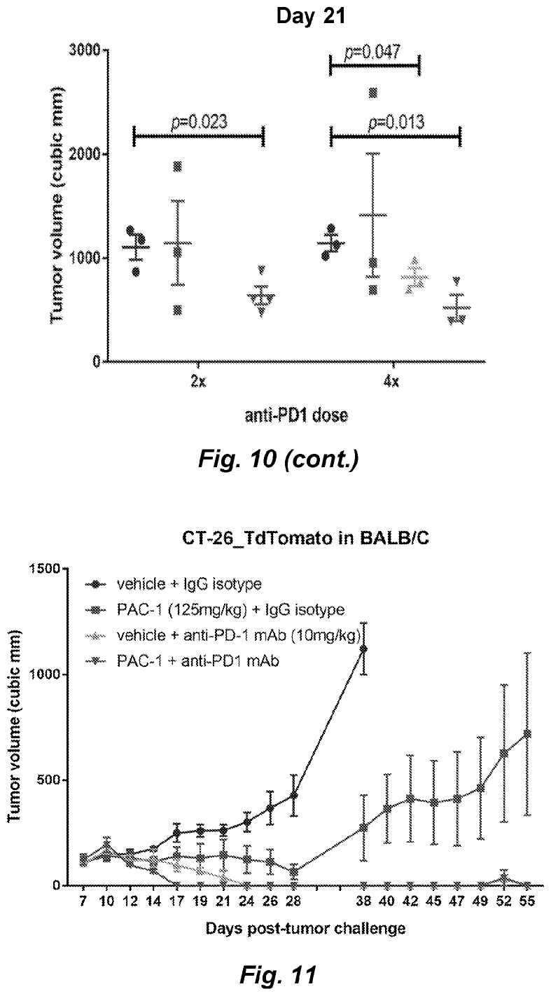

FIG. 9 illustrates that a single agent, PAC-1, exhibited a large variation in controlling sc CT-26_WT growth in BALB/c mice. Furthermore, the combination of PAC-1 and anti-PD-1 mAb significantly reduced growth of CT-26_WT in BALB/c mice compared to a vehicle+anti-IgG control. FIG. 10 illustrates a combination of PAC-1 and anti-PD-1 monoclonal antibody (mAb) reduced growth of CT-26_WT cells in BALB/c mice compared to control mice (injected with empty vehicle+anti-IgG antibody). The relative contribution of PAC-1 in this combination therapy is much more pronounced when the dosage of anti-PD-1 mAb is reduced from 4 doses to 2 doses. These experiments indicate also that days 14 and 21 are good time point during which to perform TIL analysis.

FIG. 11 illustrates the development of CT-26_TdTomato subcutaneous tumor model in BALB/c mince. Mice were inoculated with 1×10⁶ CT-26_TdTomato cells subcutaneously at their hind flank. Ten days post-inoculation (tumor volume ˜150 mm³), mice were given the following treatments:

Group 1: 3 mice—vehicle+rat IgG isotype mAb (10 mg/kg)

Group 2: 3 mice—PAC-1 (125 mg/kg)+rat IgG isotype mAb (10 mg/kg)

Group 3: 3 mice—vehicle+anti-PD1 mAb (10 mg/kg)

Group 4: 3 mice—PAC-1 (125 mg/kg)+anti-PD1 mAb (10 mg/kg)

BALB/C mice in group 4 (PAC-1+anti-PD1 mAb) were able to reject the CT-26-TdT after 5 days of consecutive PAC-1 treatment and 2× administration of anti-PD1. At day 47, the mice still appear tumor-free. The anti-PD1-treated group was able to clear tumor after 3 injections of anti-PD1 mAb. At day 47, mice still appear tumor-free. At Day47, mice that were still tumor-free were re-challenged with 1×10⁶ CT-26_TdTomato cells. No significant increase in tumor volume was observed after the re-challenge.

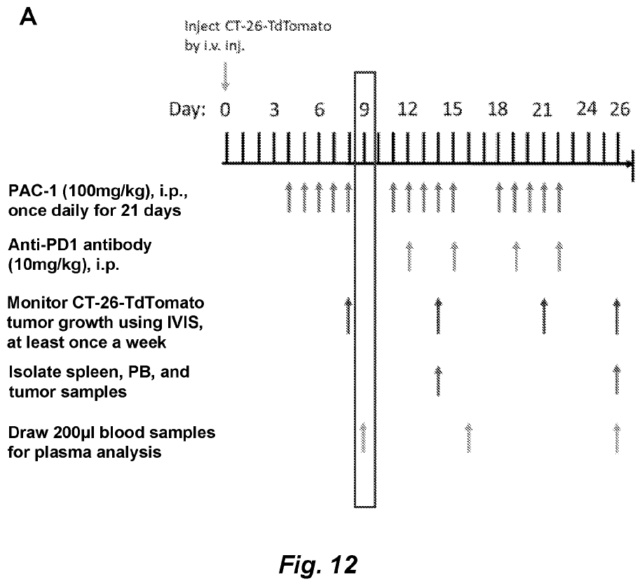

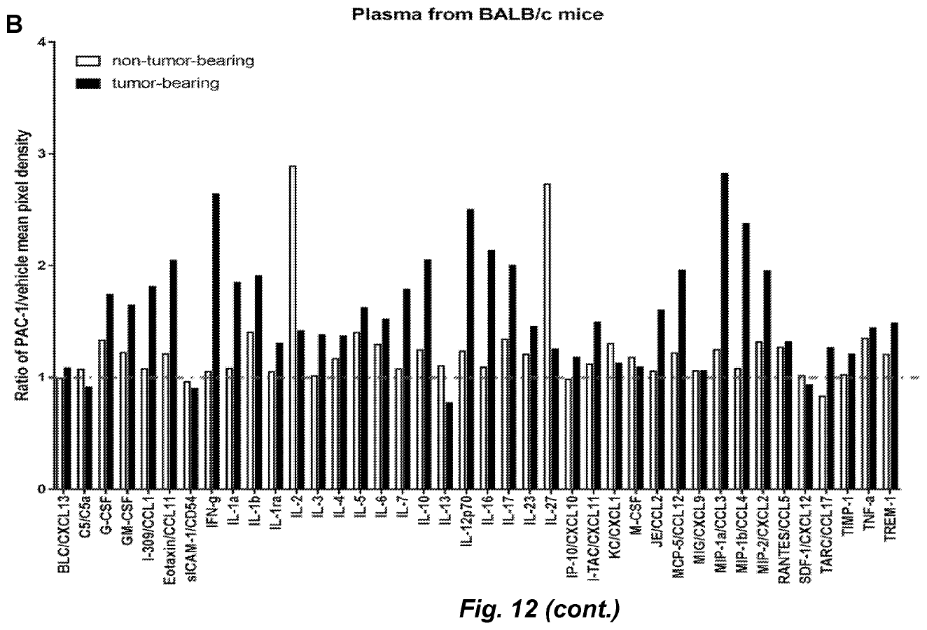

FIG. 12A illustrates a cytokine array indicating PAC-1 is immunogenic and leads to an increase in cytokines that promote macrophage differentiation as well as B-cell and T-cell proliferation. As shown in FIG. 12B, ˜100 ul blood was collected via retro-orbital blood extraction in heparinized vials. White blood cells were centrifuged at 8000 g for 10 mins, and plasma/supt was transferred to new tubes. Cytokine array was performed on 4 groups using pooled samples from 2-3 mice:

Non-tumor-bearing+vehicle

Non-tumor-bearing+PAC-1 (×5 doses)

Tumor-bearing+vehicle

Tumor-bearing+PAC-1 (×5 doses)

Signals were quantified using ImageJ. Data was normalized by calculating the ratio of PAC-1/vehicle mean pixel density as shown in the plot.

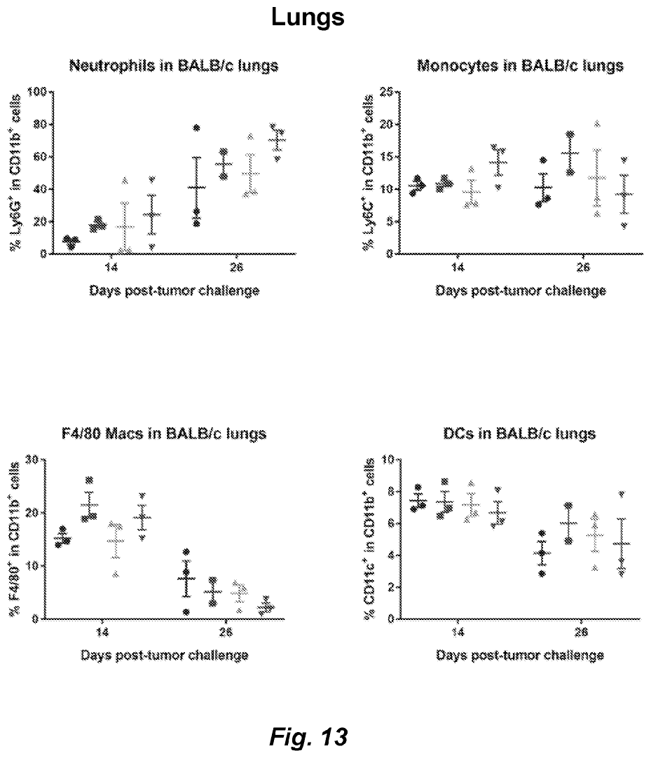

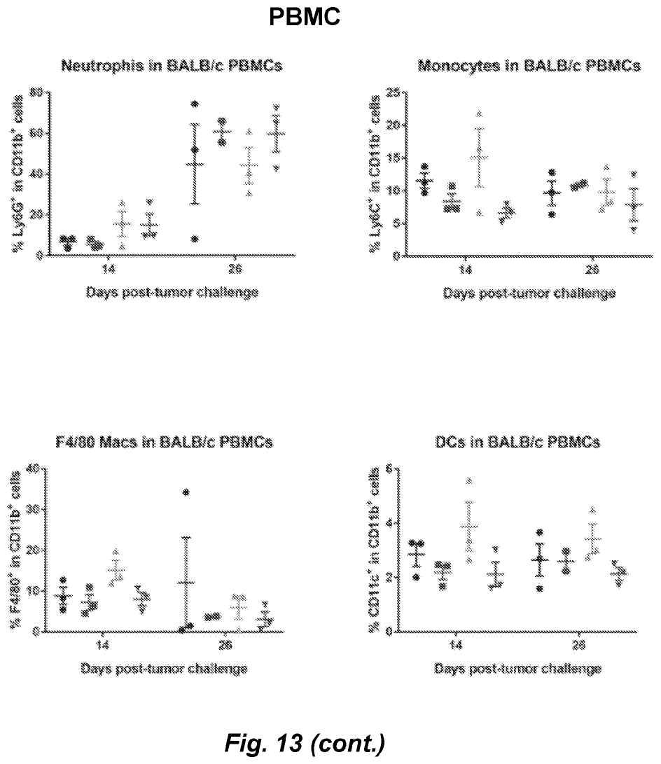

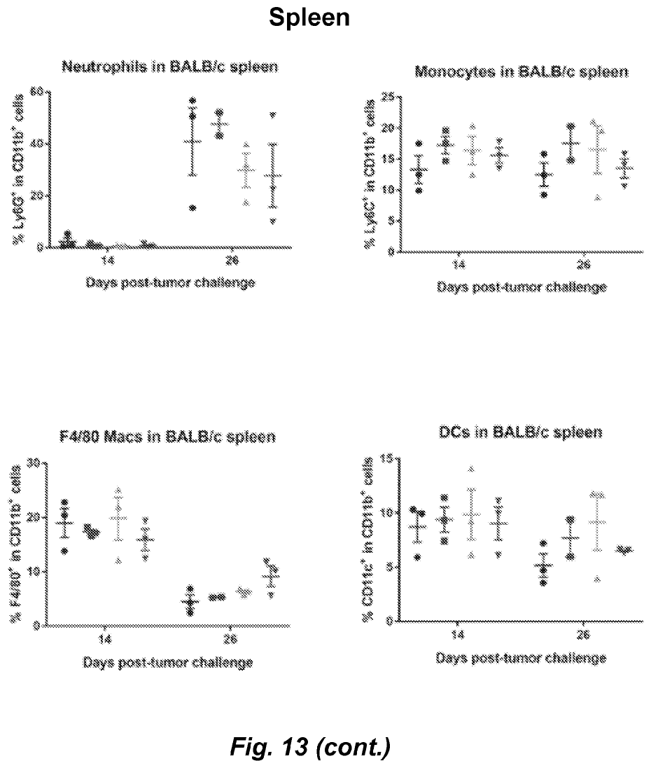

FIG. 13 illustrates that at day14 post-tumor challenge, neutrophils and macrophages appear to increase in the lung tumor microenvironment following PAC-1 treatment. At day 26, the population of macrophages and dendritic cells in the tumor microenvironment have decreased.

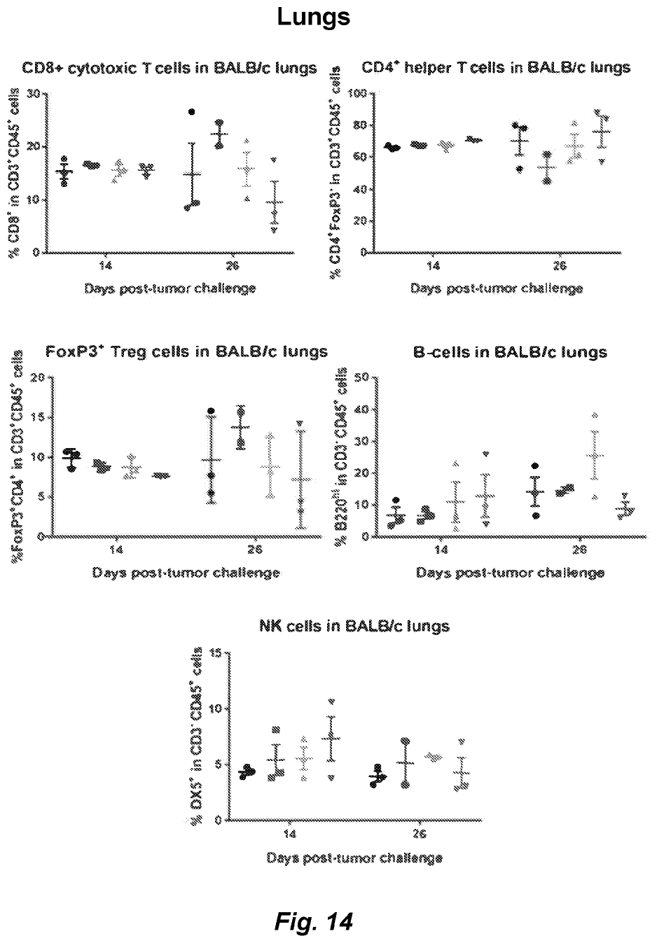

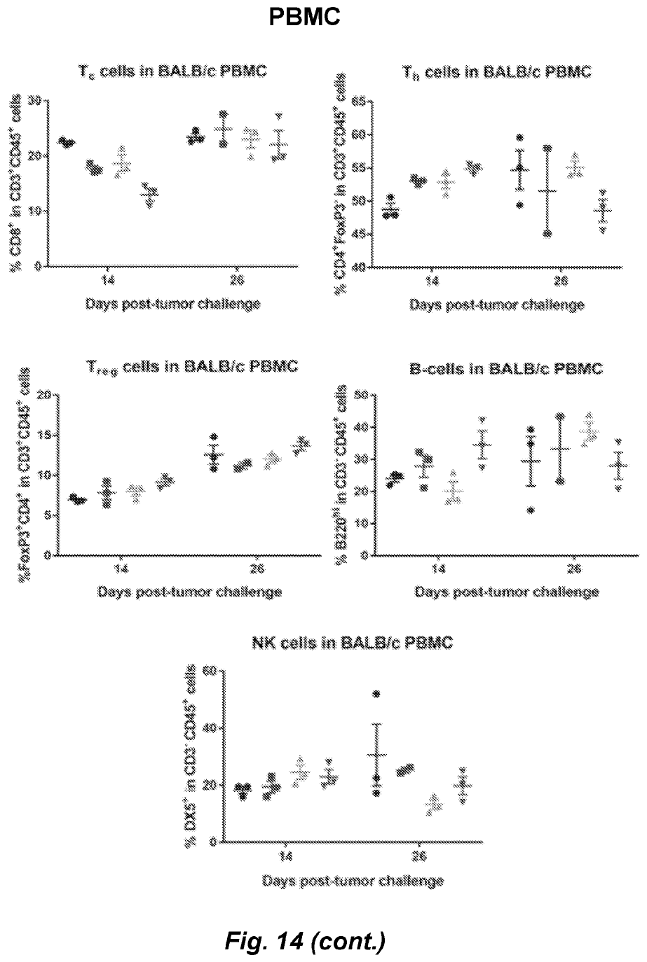

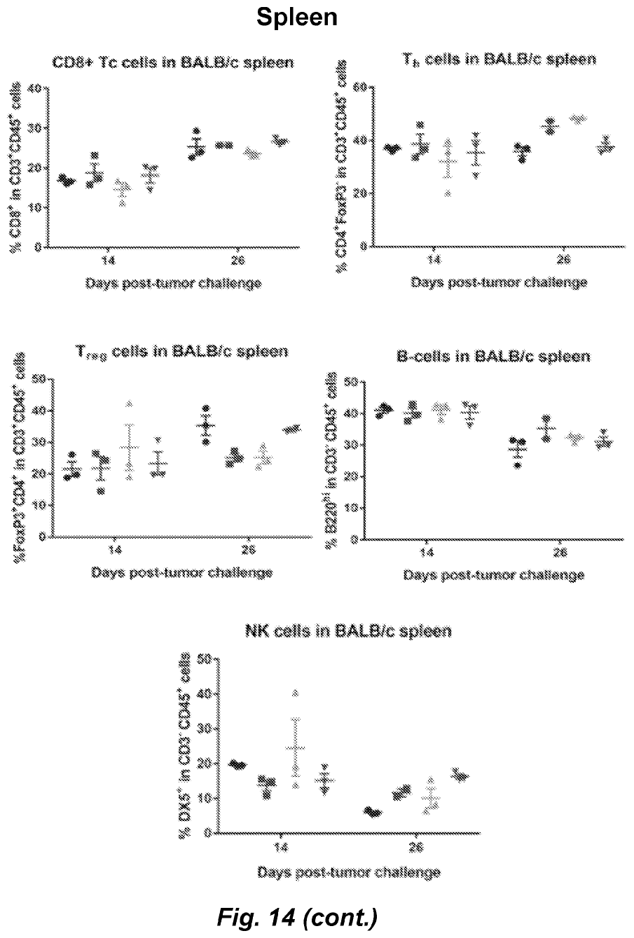

FIG. 14 illustrates CD4⁺ Tₕ cells increase in the lung tumor microenvironment on day 26 following a combinatorial PAC-1 and anti-PD-1 treatment. The percentage of FoxP3⁺ T_{regs} in the lungs was lowest in the group with combination treatment.

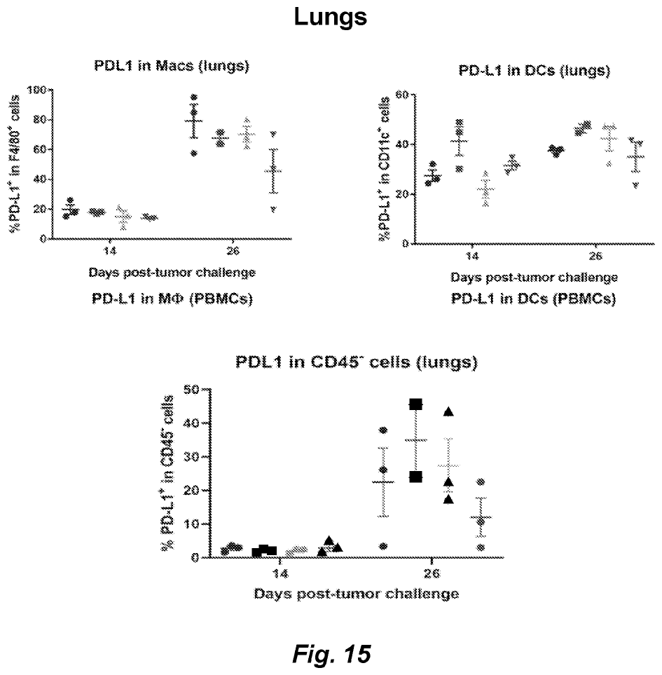

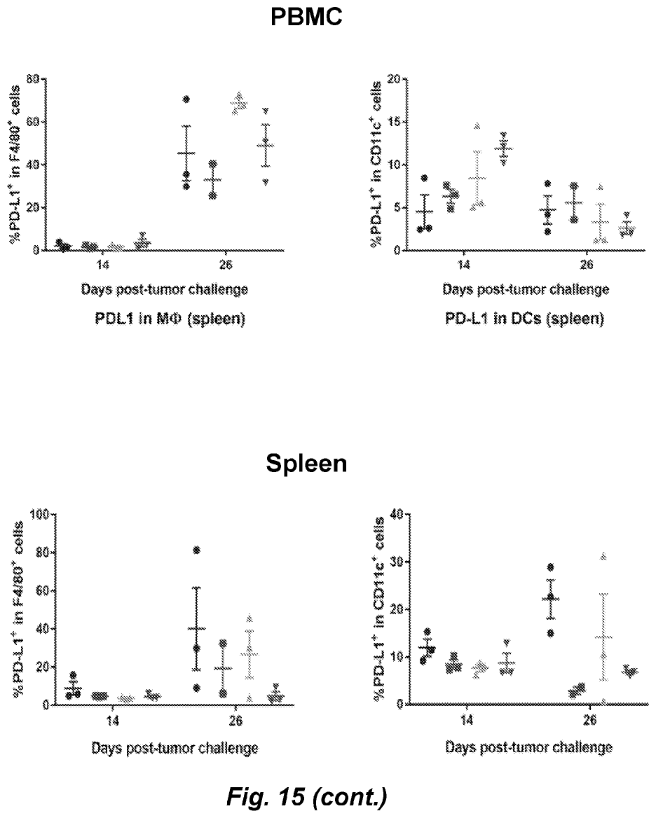

FIG. 15 illustrates PD-L1 expression on dendritic cells and CD45⁻ (tumor) cells increased on day 26 post-tumor challenge and may have contributed to T cell exhaustion.

### Example 4. PAC-1 and Immunotherapy in a Syngeneic Colon Cancer Model (MC-38 Cells)

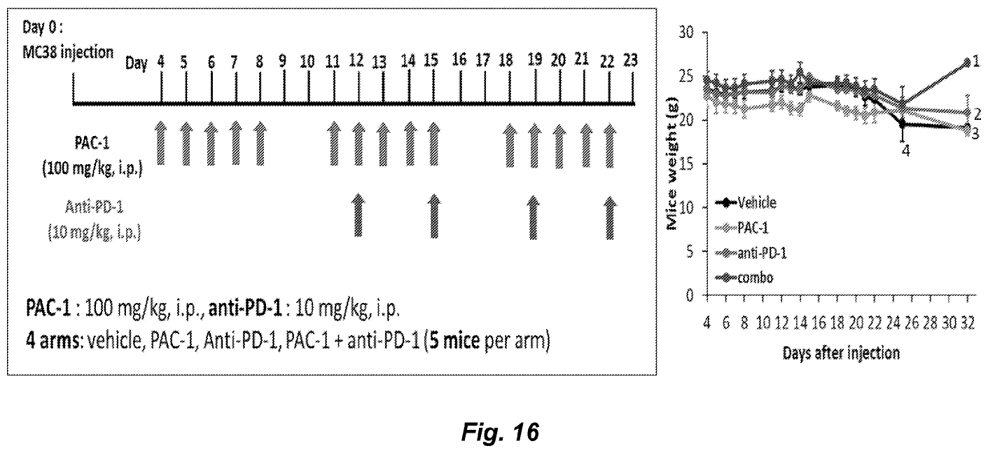

FIG. 16 illustrates the development of MC-38 metastasis model in C57BL/6 mice and treatment with PAC-1 in combination with anti-PD-1 antibody. MC-38 cells were injected via a tail vein with 1×10⁶ cells/mouse. PAC-1 was injected (i.p.) 100 mg/kg and anti-PD-1 was injected (i.p.) at 10 mg/kg over a 23-day period post MC38 injection. The weight of the mice injected with the combination of PAC-1/anti-PD-1 treatment showed significant weight recovery beginning at about day 24 and increasing until day 32.

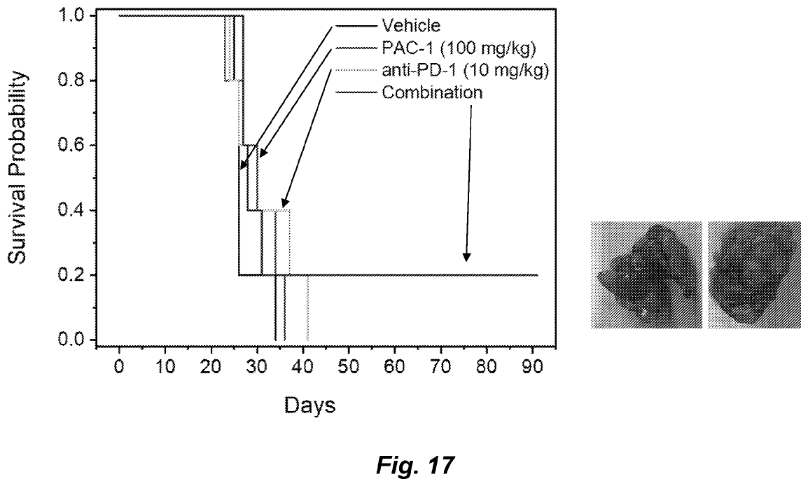

FIG. 17 illustrates a survival curve of mice after challenged with MC-38 cells and later injected with an empty vehicle control, PAC-1, anti-PD-1 antibody, or a combination of PAC-1 and anti-PD-1 antibody. These results demonstrate a steady survival probability after about 32 days for mice injected with both of PAC-1 and anti-PD-1 antibody.

### Example 5. Pharmaceutical Dosage Forms

The following formulations illustrate representative pharmaceutical dosage forms that may be used for the therapeutic or prophylactic administration of a composition of a formula described herein, a composition specifically disclosed herein, or a pharmaceutically acceptable salt thereof (hereinafter referred to as ‘Composition X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the disclosure. Changes and modifications can be made in accordance with ordinary skill in the art without departing from the disclosure in its broader aspects as defined in the following claims.

All publications, patents, and patent documents are incorporated by reference herein, as though individually incorporated by reference. No limitations inconsistent with this disclosure are to be understood therefrom. The disclosure has been described with reference to various specific and preferred embodiments and techniques. However, it should be understood that many variations and modifications may be made while remaining within the spirit and scope of the disclosure.

1. A composition comprising:
(a) a procaspase-3 activator;
(b) at least one second active agent, wherein the second active agent is a check-point inhibitor, cancer vaccine, metabolic modulator, macrophage inhibitor, or immune-stimulator or modulator; and
(c) optionally a pharmaceutically acceptable diluent, excipient, or carrier.

2. The composition of claim 1 wherein the procaspase-3 activator is PAC-1:

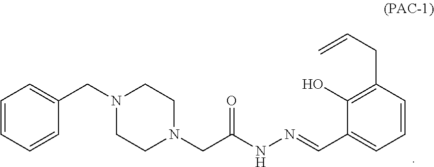 [SMILES: C=CCc1cccc(/C=N/NC(=O)CN2CCN(Cc3ccccc3)CC2)c1O]

3. The composition of claim 1 wherein the second active agent has an effect in a cancer cell that induces apoptosis and PAC-1 enhances the effect of the second active agent by an amount greater than an additive effect, wherein PAC-1 primes the vulnerability of the cancer cell to the second active agent.

4. The composition of claim 1 wherein the second active agent modulates indoleamine-pyrrole 2,3-dioxygenase (IDO), adenosine A2A receptor (A2AR), transforming growth factor beta (TGF-β), C—X—C chemokine receptor type 4 (CXCR-4), C—C chemokine receptor type 4 (CCR4), tumor necrosis factor receptor (CD27), interleukin-2 receptor subunit beta (CD122), death receptor 5 (DR5), inhibitors of apoptosis proteins (IAP), glutaminase, colony stimulating factor 1 receptor (CSF1R), toll-like receptors (TLRs), dendritic cells (DC), or a combination thereof.

5. The composition of claim 1 wherein the second active agent is ADXS11-001, ADXS31-142, AMP-224, AMP-514, atezolimumab, atezolizumab, avelumab, bevacizumab, cemiplimab, BLZ945, BMS-936559, BMS986016, BMS986156, BMS986205, CB839, CIMAvax, CMP001, CP870893, CPI-444, CRS207, CV301, DC vaccine, DNX2401, DS-8273a, durvalumab, epacadostat, FAZ053, FPA008, GDC0919, GSK3174998, GVAX, GWN323, IMCgp100, IMP321, imprime PGG, indoximid, ipilimumab, JTX-2011, LAG525, LCL161, LK-301, LY2157299, LY2510924, LY3022855, MBG453, MEDI0562, MEDI0680, MEDI6469, MEDI9447, MGN1703, mogamulizumab, MOXR0916, neoantigen vaccine, NEO-PV-01, NIS793, nivolumab, NKTR-214, PBF509, PDR001, pembrolizumab, peptide vaccine, pexidartinib (PLX3397), PF-04518600, PF-3512676, REGN2810, REGN3767, R07009789, SD101, talimogene laherparepvec, TPIV200/huFR-1, tremelimumab, TroVax, TSR022, ulocuplumab, urelumab, utomilumab, varlilumab, viagenpumatucel-L (HS-110), or a combination thereof.

6. The composition of claim 1 wherein the at least one second active agent is at least one check-point inhibitor that regulates an immune response via programmed cell death protein 1 (PD-1), programmed death-ligand 1 (PD-L1), cytotoxic T-lymphocyte-associated protein 4 (CTLA-4), T-cell immunoglobulin and mucin-domain containing-3 (TIM-3), lymphocyte-activation gene 3 (LAG-3), tumor necrosis factor receptor superfamily-member 4 (TNFRSF4 or OX40), tumor necrosis factor receptor superfamily-member 9 (TNFRSF9 or 4-1BB), glucocorticoid-induced TNFR-related protein (GITR), inducible T-cell costimulator (ICOS), or a combination thereof.

7. The composition of claim 6 wherein the checkpoint inhibitor is anti-PD-1, anti-CTLA-4, or a combination thereof; wherein the anti-PD-1 is nivolumab or pembrolizumab, the anti-CTLA-4 is ipilimumab or tremelimumab, or a combination thereof.

8. The composition of claim 1 wherein the concentration of PAC-1 is about 0.1 μM to about 50 μM and the concentration of the second active agent is about 1 nM to about 100 μM.

9. The composition of claim 1 comprising a pharmaceutically acceptable diluent, excipient, or carrier, wherein a) the carrier comprises water, a buffer, a sugar, a cellulose, a cyclodextrin, dimethyl sulfoxide, polyethylene glycol, tocopherol, a liposome, a micelle, or a combination thereof, or b) the excipient comprises, a binder, a lubricant, a sorbent, a vehicle, a disintegrant, a preservative, or a combination thereof.

10. The composition of claim 1 wherein the composition selectively targets cancer cells, wherein the cancer cells are cells of bladder cancer, breast cancer, colon cancer, endometrial cancer, glioblastoma, leukemia, liver cancer, lung cancer, lymphoma, melanoma, meningioma, multiple myeloma, ovarian cancer, osteosarcoma, pancreatic cancer, prostate cancer, renal cancer, or thyroid cancer;
wherein the breast cancer is optionally triple negative breast cancer, lung cancer is optionally non-small cell lung cancer, and renal cancer is optionally metastatic renal cell carcinoma.

11. A method of inhibiting the growth or proliferation of cancer cells comprising contacting cancer cells with an effective amount of a composition of claim 1, thereby inhibiting the growth or proliferation of the cancer cells.

12. The method of claim 11 wherein the growth or proliferation of the cancer cells is inhibited by suppressing mismatch-repair (MMR) proteins, or by caspase-3 activation mediated degradation of MutL homolog 1 (MLH1) proteins;
wherein DNA microsatellite instability (MSI) is induced.

13. A method of inducing apoptosis in a cancer cell comprising contacting the cancer cell with an effective amount of a composition of claim 1, wherein apoptosis is induced by suppressing mismatch-repair (MMR) proteins in the cancer cell.

14. The method of claim 13 wherein the MMR proteins are MutL homolog 1 (MLH1) proteins, wherein degradation of MLH1 proteins, mediated by caspase-3 activation via the procaspase-3 activator, induces apoptosis in the cancer cell.

15. A method of treating a cancer comprising administering to a subject in need thereof, concurrently or sequentially, a therapeutically effective amount of a procaspase-3 activator and an effective amount of a second active agent, wherein the second active agent is an immunotherapeutic, wherein the effect of the immunotherapeutic is enhanced by the administration of the procaspase-3 activator.

16. The method of claim 15 wherein the procaspase-3 activator is PAC-1:

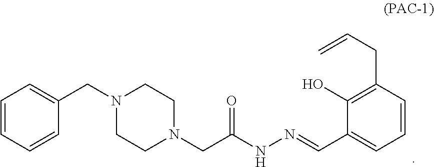 [SMILES: C=CCc1cccc(/C=N/NC(=O)CN2CCN(Cc3ccccc3)CC2)c1O]

17. The method of claim 16 wherein the concentration of PAC-1 is about 0.1 μM to about 50 μM and the concentration of the second active agent is about 1 nM to about 100 μM.

18. The method of claim 16 wherein the concentration of PAC-1 is about 1 μM to about 10 μM and the concentration of the second active agent is about 1 nM to about 1 μM; or
wherein the total administered dose per day of PAC-1 is about 10 mg/kg to about 125 mg/kg and the daily total administered dose per day of the second active agent is about 1 mg/kg to about 100 mg/kg.

19. The method of claim 15 wherein the second active agent comprises a check-point inhibitor, cancer vaccine, metabolic modulator, macrophage inhibitor, immune-stimulator, or modulator; or a combination thereof.

20. The method of claim 15 wherein the second active agent is atezolimumab, avelumab, bevacizumab, BMS986016, BMS986156, CP870893, durvalumab, FAZ053, GSK3174998, GWN323, IMP321, ipilimumab, JTX-2011, LAG525, MBG453, MEDI0562, MEDI0680, MEDI6469, MOXR0916, nivolumab, PDR001, pembrolizumab, PF-04518600, REGN2810, REGN3767, R07009789, tremelimumab, TSR022, urelumab, utomilumab, or a combination thereof.

21. The method of claim 15 wherein the immunotherapeutic is a check-point inhibitor, and the check-point inhibitor regulates an immune response via programmed cell death protein 1 (PD-1), programmed death-ligand 1 (PD-L1), cytotoxic T-lymphocyte-associated protein 4 (CTLA-4), T-cell immunoglobulin and mucin-domain containing-3 (TIM-3), lymphocyte-activation gene 3 (LAG-3), tumor necrosis factor receptor superfamily-member 4 (TNFRSF4 or OX40), tumor necrosis factor receptor superfamily-member 9 (TNFRSF9 or 4-1BB), glucocorticoid-induced TNFR-related protein (GITR), inducible T-cell costimulator (ICOS), or a combination thereof.

22. The method of claim 15 wherein the procaspase-3 activator suppresses mismatch-repair (MMR) proteins;
wherein the MMR proteins comprise MutL homolog 1 (MLH1) proteins, and wherein degradation of MMR proteins, mediated by caspase-3 activation via the procaspase-3 activator, induces a deficiency in MMR proteins, DNA microsatellite instability (MSI), neoantigen expression, or a combination thereof, thereby enhancing the effect of the immunotherapeutic, and wherein the procaspase-3 activator increases tumor-infiltrating lymphocytes in the cancer.

23. The method of claim 15 wherein the cancer is bladder cancer, breast cancer, colon cancer, endometrial cancer, glioblastoma, leukemia, liver cancer, lung cancer, lymphoma, melanoma, meningioma, multiple myeloma, ovarian cancer, osteosarcoma, pancreatic cancer, prostate cancer, renal cancer, or thyroid cancer;
wherein the breast cancer is optionally triple negative breast cancer, lung cancer is optionally non-small cell lung cancer, and renal cancer is optionally metastatic renal cell carcinoma.

24. The method of claim 15 wherein:
the compound PAC-1 and the second active agent are concurrently administered to the subject; or
the compound PAC-1 and the second active agent are sequentially administered to the subject, wherein the compound PAC-1 is administered to the subject before the second active agent or the compound PAC-1 is administered to the subject after the second active agent.

25. The method of claim 15 wherein the compound PAC-1 and the second active agent are administered to the subject once daily (q.d.), twice a day (b.i.d.), three times a day (t.i.d.), or four times a day (q.i.d.), wherein the total administered dose per day of PAC-1 is about 1 mg/kg to about 150 mg/kg; or
each administered dose of PAC-1 is about 70 mg, about 175 mg, about 250 mg, about 375 mg, about 450 mg, about 500 mg, about 625 mg, about 750 mg, or about 1000 mg; or
each administered dose of PAC-1 is about 50 mg/m² to about 250 mg/m².

26. A method of treating a cancer comprising administering to a subject in need thereof, PAC-1 and an anti-PD-1 antibody wherein the PAC-1 is administered daily for 21 or more consecutive days such that a total administered dose per day of the PAC-1 is about 100 mg/kg to about 125 mg/kg and the anti-PD-1 antibody is administered two times or four times over the 21 or more consecutive days, wherein a dose of the anti-PD-1 antibody is about 10 mg/kg and each of the dose of the anti-PD-1 antibody is administered on separate days.
